(19) United States
(12) Patent Application Publication
(10) Pub. No.: US 2021/0264596 A1
(43) Pub. Date: Aug. 26, 2021

(54) SYSTEM AND METHOD FOR PRODUCING A MULTIPARAMETER GRAPHIC INDICATOR FROM AN IMAGE OF A HISTOLOGICAL SECTION

(71) Applicant: BIOCELLVIA, Marseille (FR)

(72) Inventors: Yvon JULE, Marseille (FR); Florent TOMI, Marseille (FR); Jean-Claude GILHODES, Marseille (FR)

(73) Assignee: BIOCELLVIA, Marseille (FR)

(21) Appl. No.: 17/256,316

(22) PCT Filed: Jun. 28, 2019
(86) PCT No.: PCT/FR2019/051614

§ 371 Date: Dec. 28, 2020

(30) Foreign Application Priority Data
Jun. 29, 2018 (FR) 1855953

(51) Int. Cl. G06T 7/00; G06T 7/62; G06T 11/20; G16H 30/40; G16H 50/50; G16H 50/30; G16H 50/70; G16H 15/00

(52) U.S. Cl. CPC G06T 7/0012; G06T 7/62; G06T 11/206; G16H 30/40; G16H 50/20; G16H 50/30; G16H 50/70; G16H 15/00; G06T 2207/30061; G16H 50/50

(57) ABSTRACT

A method for producing a multiparameter graphic indicator relating to a remodelling of the human or animal bronchial epithelium from a digital representation of a histological section of a lung comprising one or more components, each of which describes a wall enclosing a lumen. Such a method makes it possible, in particular, to estimate one or more quantities of interest relating to one or more components in order to produce a graphical representation describing the quantity or quantities of interest so as to assist with diagnosing a disease or analyzing the effectiveness of a molecule for therapeutic purposes. The method is implemented by a processing unit of an electronic object of a histological analysis system.

The invention relates to a system and a method for producing a multiparameter graphic indicator with reference to the remodelling of the epithelium of a tissue of a human or animal organ, from an image of a histological section, and thus delivering an objective and reproducible aid to healthcare personnel so that they can establish a diagnosis with reference to a possible human or animal pathology. Moreover, the invention provides an objective and reproducible aid so that an investigator in the laboratory can estimate the curative relevance of a given treatment with respect to such a pathology.

Biological imaging is currently one of the major resources in exploration of the organs and the different organic tissues. In particular, it is predominantly involved in the fields of medical diagnosis support and preclinical and clinical research.

Different techniques are currently utilized in preclinical and clinical imaging, such as, non-limitatively, magnetic resonance imaging, optical, electronic and confocal microscopy, microtomography, ultrasound scanning, CT scanning. These techniques can be utilized for in vivo or ex vivo observations. The digital images thus obtained make it possible, in the context of institutional or industrial research laboratories, more particularly to analyze a biological state of organic tissues and to assess certain beneficial and/or toxic effects of certain substances with a view to the selection thereof for the development of future medicinal products.

In the era of digital transformation, the development of these digital imaging technologies has opened new perspectives for histological analysis overall.

The ability to access digital images of histological sections has made it possible to develop new methods based on descriptive and quantitative analysis of the digital images of said histological sections, by means of computerized tools utilizing innovative algorithms or innovative methods allowing an advance in terms of precision, reliability, speed and reproducibility.

However, utilization of the computerized tools that are currently available does not make it possible to automate the quantitative assessment of certain pathologies, such as, by way of non-limitative example, respiratory tract disorders. In fact, the investigator still remains only too present in the process of implementation of this assessment. Their manual in-person intervention leads to wide variabilities in characterization of the components of the samples of histological slides assessed.

In the context of aid in the diagnosis of certain pathologies affecting the respiratory tract, more particularly the small airways, such as for example small airway remodelling, also known by the acronym SAR, no conventional assessment exists for this type of respiratory disorder. Each laboratory carries out its own measurements from its own reference information, which most often relate to some morphometric measurements, such as bronchial perimeters and/or diameters. These measurements are carried out manually by an investigator from optical microscopy images.

Other techniques are also found making it possible to identify and quantify a degree of severity of a pathology linked to SAR. Some use histological sections, others are used in vivo. Among these, there may be mentioned spirometry in particular. This first technique, which is known and widely used both in diagnosis and in determining the degree of severity of pulmonary diseases, consists of testing pulmonary function. A diagnosis relating to an obstructive pulmonary disease arises when a ratio between a forced expiratory volume in one second (FEV1) and a forced vital capacity (FVC) is less than seventy percent. Although a reduction of the FEV1 can reflect an obstruction of the airflow, nevertheless it also depends on lung volumes, the lung elastic recoil phenomenon, respiratory muscle strength and/or the effort of the patient. This method thus has significant limitations with regard to the use of the parameters mentioned above, the variation of which can be induced by pathologies other than SAR. Such limitations are also present in the context of the diagnosis of a less severe SAR, wherein diagnosis by such a method can prove difficult.

A second technique consists of estimating lung volumes by plethysmography. It delivers a sensitive measurement of the trapped gases and lung hyperinflation, which can be defined as an abnormal elevation of lung volumes at the end of exhalation. This measurement gives a function of the airflow limitation, the lung elastic recoil phenomenon and the chest wall compliance of a patient. Airway stenosis in fact leads to an increase of the exhalation time required to evacuate all of the air contained in the lungs. Thus it may be that the respiratory tract closes, thus trapping the remaining gas. The residual volume (RV) of remaining gas also consists of a measurement indicating a dysfunction of the small respiratory airways. Said volume can be directly correlated with the degree of morphological changes of the respiratory tract caused by inflammations present in the small airways. A ratio of said residual volume to the total lung capacity (TLC), which can be denoted RV/TLC, can easily be established by this method, as can an airway resistance which increases during obstructive pulmonary disorders. However, this indicator is not specific to the small respiratory airways, which limits application thereof in diagnosis and monitoring of diseases of the SAR type.

A third technique can also be used, known as “single-photon emission computed tomography” or “SPECT”. Such a three-dimensional imaging technique consists of using several gamma-ray detectors which rotate around a recumbent patient. A reconstruction of images obtained thus makes it possible to visualize a radionuclide distribution in three dimensions, thus offering an assessment of the ventilation of different pulmonary regions.

Although currently very widely used, these three measurement methods have a number of drawbacks. Firstly, they require a lengthy implementation time, i.e. sometimes several hours, and are dependent on the investigator who utilizes them. Thus generally they involve an additional analysis by a specialist pathologist in order to confirm or rule out the first results. This is particularly the case for spirometry. Involvement of several investigators or specialist personnel thus results in a significant variability in the results relating to the assessment of a pathology, and thus diagnoses that are sometimes random or contradictory.

Moreover, the SPECT analysis technique can entail a major drawback and a health risk for a patient during the investigation of pathologies, in view of the radiation applied to said patient. Limitations as to the intensity of said radiation and/or to the acquisition time of the experimental signals can impact the assessment of the radionuclide removal after deposition thereof in different pulmonary regions of interest, thus hampering a relevant assessment of the ventilation of said pulmonary regions of the patient.

Thus, the invention makes it possible to overcome all or part of the drawbacks raised above and makes it possible to provide invaluable aid to any investigator wishing to estimate quantities of interest with a view to producing a graphic indicator in order to facilitate the establishment of a diagnosis with reference to a human or animal pathology, or to assess the relevance of a treatment with respect to said pathology.

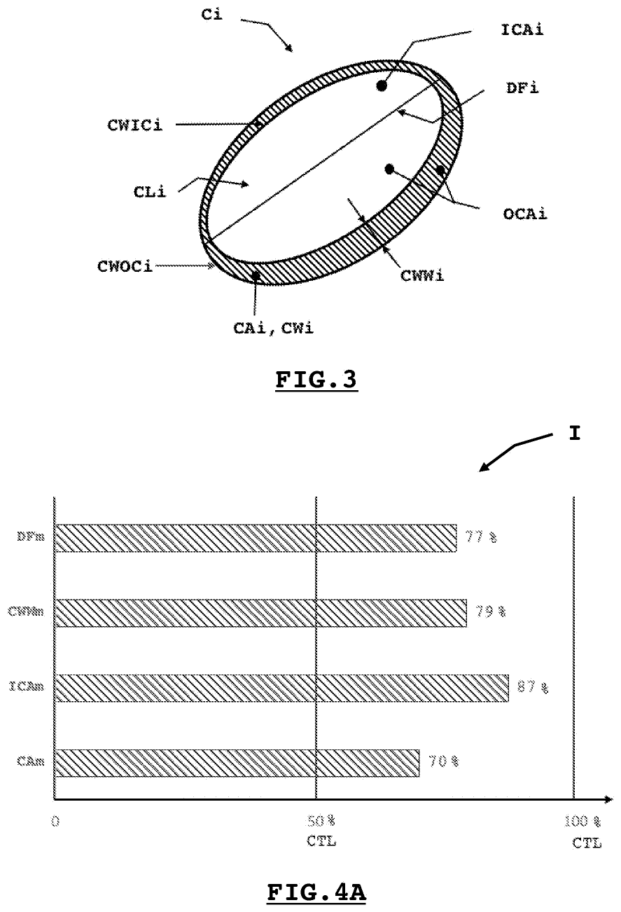

Certain chronic pulmonary diseases are characterized by remodelling of the pulmonary tissues (SAR) affecting the alveolar parenchyma, the bronchi and the vasculature. The invention allows a very accurate quantitative morphometric analysis of the components observed in a histological section, thus making it possible to characterize, for example, a remodelling of the respiratory tracts, and thus makes it possible to deliver a multiparameter graphic indicator with reference to the morphology of tubular components of an organ, in particular the lungs, and more particularly the small respiratory airways. The invention makes use of a digital representation of a histological section of said organ, describing a plurality of annular components resulting from the sectioning of said tubular components in order to perform a histological section. Said tubular components are evidenced by means of annular components in a two-dimensional representation, on completion of a histological section. The concept of tubular component will be described in greater detail hereinafter, with reference to FIG. 3.

Among the numerous advantages achieved by the invention, there may be mentioned that it makes it possible to:
reduce considerably the analysis time necessary for establishing a diagnosis of a pathology by an investigator, reducing said time to less than one minute according to the processing power of the device or of the electronic system implementing a method according to the invention;
increase the accuracy and reliability of the measurements of an analyzed sample;
overcome the variability of results between different investigators, delivering objective and reproducible results;
dispense with costly materials and/or methods that are harmful or invasive for the patients examined.

To this end, the invention relates to a method for producing a multiparameter graphic indicator relative to a remodelling of the human or animal bronchial epithelium from a digital representation in the form of a matrix of a determined number of pixels, of a histological section of a lung comprising one or more components having annular shape, each one of which describes a wall encircling a lumen, the method being implemented by a processing unit of a system for histological analysis, said system comprising moreover a human-machine interface output and a data memory.

In order to be able to produce a multiparameter graphic indicator, said method comprises:
a step for estimating, from pixels of said digital representation of a histological section of a lung, quantities of interest relative to the respective morphologies of the components identified in said digital representation of the histological section, said quantities of interest belonging to a set of quantities of interest comprising:
   i. the Feret diameter of the outer contour of the wall of a component;
   ii. a mean distance separating said outer contour from the inner contour, revealing a mean thickness of the wall of a component;
   iii. the area described by said inner contour of a component, revealing the lumen thereof;
   iv. the area described by said outer contour of a component, revealing the total area covered thereby;
   v. the area of the wall of a component defined by subtracting the area described by said inner contour from the area described by said outer contour of said component;
a step for producing, per estimated quantity of interest, a graphic representation thereof relative to a standard quantity of interest;
a step for causing the joint graphic outputting of the graphic representations of said quantities of interest relative to the respective standard quantities of interest produced beforehand, by the output human-machine interface of the system.

Advantageously, the step for estimating quantities of interest relative to the respective morphologies of the identified components in said digital representation of the histological section can comprise a step for registering, in the data memory, a data structure associated with each component, said data structure comprising a field for storing the value of each estimated quantity of interest.

In a variant or in addition, in order to produce a multiparameter graphic indicator relating to one or more quantities of interest of one or more types of components, the method can comprise a step for characterizing a type of component based on the value of one of the estimated quantities of interest, the step for registering in the data memory a data structure associated with each estimated quantity of interest of a component can consist of registering in a field of said data structure a value characterizing a determined type of component.

Preferentially but non-limitatively, in order to facilitate the typing of the components present in a digital representation of a histological section, when one of the estimated quantities of interest consists of the Feret diameter of the outer contour of the wall of the component, the step for characterizing a type of component of a method according to the invention can comprise an operation of comparison of the value of said estimated Feret diameter to a high-diameter threshold and/or a low-diameter threshold.

In a variant or in addition, in order to facilitate the typing of the components present in a digital representation of a histological section, when one of the estimated quantities of interest consists of the mean thickness of the wall of the component, the step for characterizing a type of component of a method according to the invention can comprise an operation of comparison of the value of said mean thickness to a predetermined high-thickness threshold and/or a low-thickness threshold.

In a variant or in addition, in order to facilitate the typing of the components present in a digital representation of a histological section, when one of the estimated quantities of interest consists of the area described by the inner contour of the component, the step for characterizing a type of component of a method according to the invention can comprise an operation of comparison of the value of said area to a predetermined high-area threshold and/or low-area threshold.

In order to provide an investigator with information relative to the morphology of one or more components of interest, it is possible for the step for producing, per estimated quantity of interest, a graphic representation thereof relative to a standard quantity of interest and/or the step for producing the joint graphic rendering of the graphic representations of said quantities of interest relative to the standard quantities of interest to be implemented for a determined characterized type of component only.

Advantageously, in order to allow an investigator to identify a remodelling of the bronchial epithelium or any other modification of the morphology of the small airways, a method according to the invention intends that the type of characterized component determined can be a bronchiolus.

Preferentially but non-limitatively, in order to provide an investigator with a visual aid making it possible instantly to compare estimated quantities of interest revealing a possible change of morphology of the components wherein said estimated quantities of interest originated, with a view to directing the diagnosis of a pathology, the step for causing the joint graphic outputting of the graphic representations of said quantities of interest relative to the respective standard quantities of interest, produced beforehand by the output human-machine interface of the system, can consist of the display by the latter of a radar chart. Such a radar chart can describe at least three graphic representations of quantities of interest relative to the respective standard quantities of interest on normed axes.

In a variant or in addition, in order to provide an investigator with a visual aid making it possible instantly to compare estimated quantities of interest revealing a possible change of morphology of the components from which said estimated quantities of interest originated, with a view to directing the diagnosis of a pathology, the step for causing the joint graphic outputting of the graphic representations of said quantities of interest relative to the respective standard quantities of interest produced beforehand by the output human-machine interface of the system, can consist of the display by the latter of a bar chart, showing the graphic representations of quantities of interest relative to the respective standard quantities of interest by normalized bars.

So as to return to an investigator a multiparameter graphic indicator having quantities of interest at one and the same scale and thus provide an instant visual aid, normalizing an axis or a bar of the step to produce the joint graphic outputting of the graphic representations of said quantities of interest relative to the respective standard quantities of interest produced beforehand by the output human-machine interface of the system can consist of expressing the value of an estimated quantity of interest as a percentage of the value of the associated standard quantity of interest.

In a variant or in addition, in order to allow an investigator instantly to visualize a significant difference between estimated quantities of interest and standard quantities of interest that are very similar, normalizing an axis or a bar of the step to produce the joint graphic outputting of the graphic representations of said quantities of interest relative to the respective standard quantities of interest produced beforehand by the output human-machine interface of the system can consist of expressing the value of an estimated quantity of interest relative to the value of the associated standard quantity of interest in the form of three predetermined values respectively describing estimated quantities of interest that are substantially lower, similar to, or greater than the values of the associated standard quantities of interest.

Moreover, according to a second subject, the invention relates to an electronic object of a system for histological analysis, said electronic object comprising a processing unit and cooperating with an output human-machine interface and with a data memory, said data memory comprising:
a digital representation of a histological section of a human or animal organ;
instructions that can be executed or interpreted by the processing means, wherein the interpretation or execution of said instructions by said processing means causes the implementation of a method according to the first subject of the invention.

According to a third subject, the invention also relates to a system for histological analysis comprising an electronic object according to the second subject of the invention and an output human-machine interface suitable for outputting to a user a multiparameter graphic indicator according to a method according to the first subject of the invention and implemented by said electronic object.

According to a fourth subject, the invention finally relates to a computer program product comprising one or more instructions that can be interpreted or executed by an electronic object according to the second subject of the invention, the interpretation or execution of said instructions by said processing unit causing the implementation of a method according to the first subject of the invention.

Other characteristics and advantages will become clearer on reading the following description and on examining the figures accompanying it, in which:

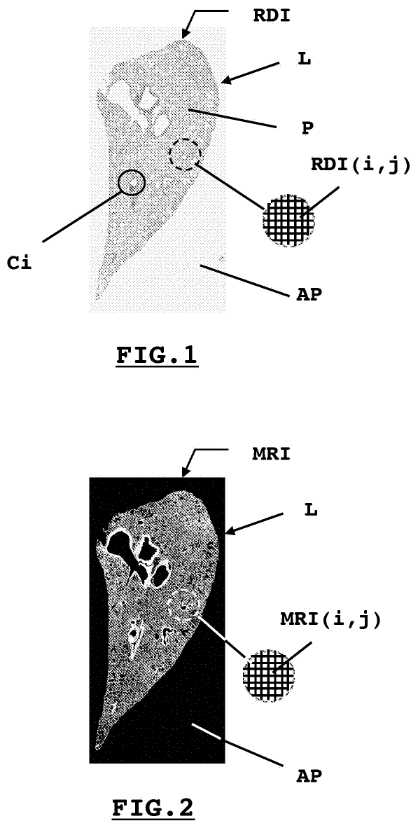

FIG. 1 presents a first digital representation of a histological section of a lung of a patient suffering from SAR, said patient being in this case a mouse;

FIG. 2 shows a second binary digital representation, originating from that shown in FIG. 1, said second binary digital representation highlighting pixels of interest with respect to other pixels;

FIG. 3 shows diagrammatically a section of a tubular component, from which are estimated quantities of interest relative to the morphology of an annular component resulting from a section of a tubular component;

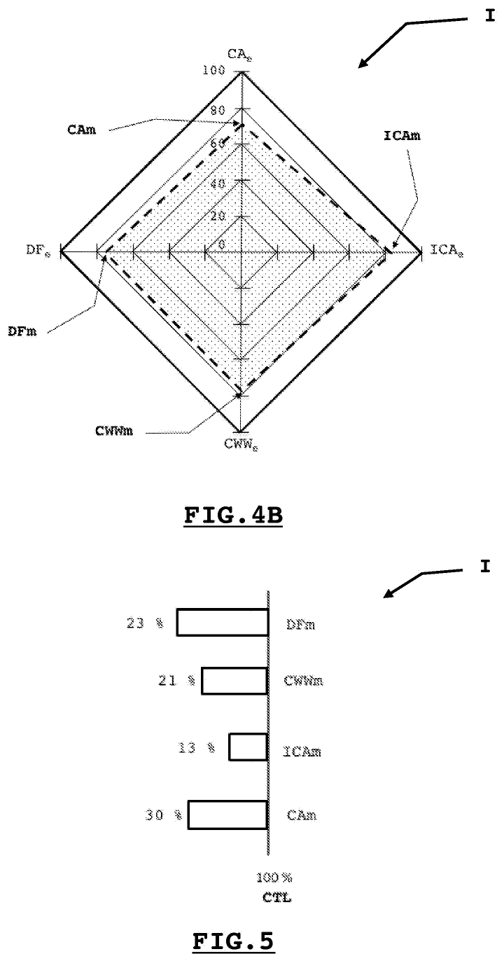

FIG. 4A presents a first example of graphic representation in the form of a bar chart, of a multiparameter graphic indicator produced by a method according to the invention, said indicator describing a plurality of estimated quantities of interest relative to a plurality of respective standard quantities of interest on normed axes, in a patient suffering from acute SAR;

FIG. 4B presents a second example of graphic representation, in the form of a radar chart, of a multiparameter graphic indicator produced by a method according to the invention, said indicator showing a plurality of estimated quantities of interest relative to a plurality of respective standard quantities of interest on normed axes, in a patient suffering from acute SAR;

FIG. 5 presents a first example of graphic representation in the form of a bar chart, of a multiparameter graphic indicator produced by a method according to the invention, said indicator describing a plurality of estimated quantities of interest relative to a plurality of respective standard quantities of interest on normed axes, in a patient suffering from acute SAR;

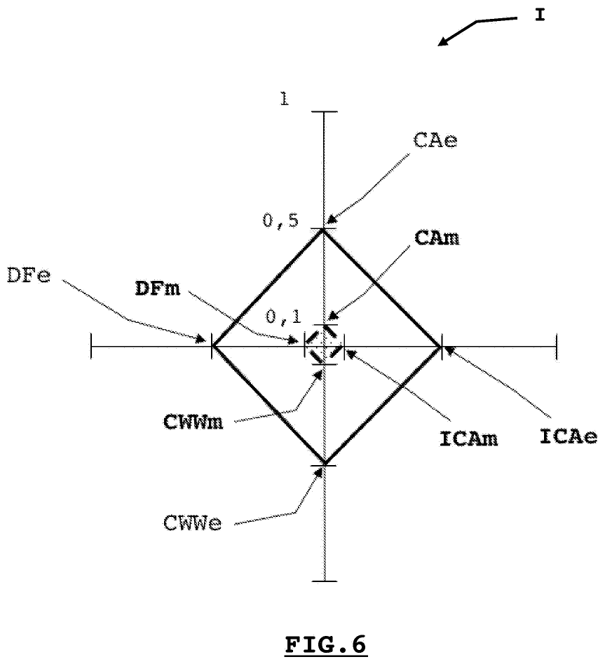

FIG. 6 presents a second example of graphic representation, in the form of a radar chart, of a multiparameter graphic indicator produced by a method according to the invention, said indicator showing a plurality of estimated quantities of interest relative to a plurality of respective standard quantities of interest on normed axes, in a patient suffering from acute SAR;

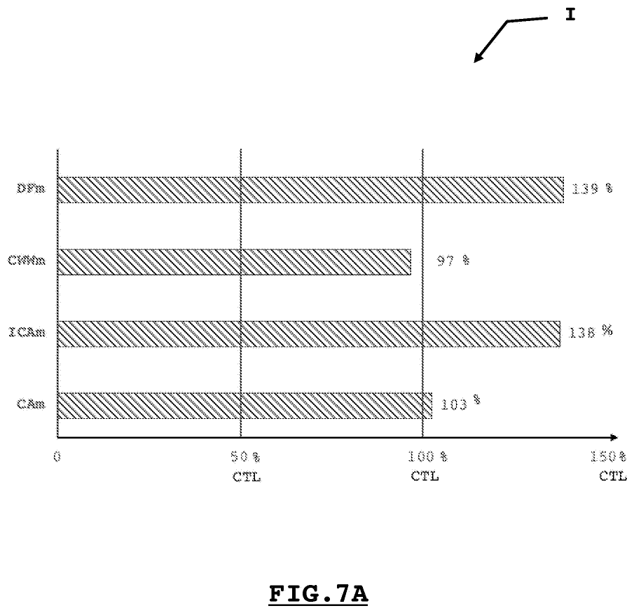

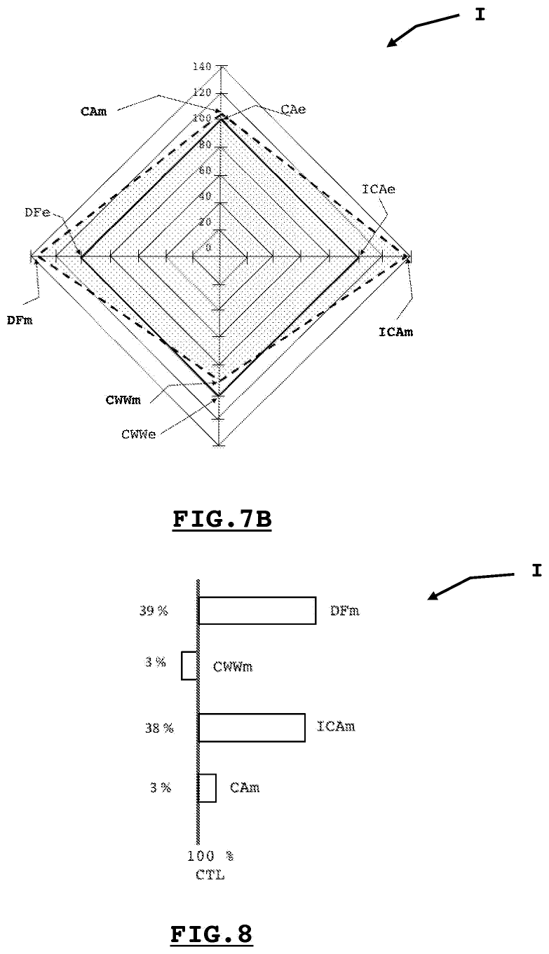

FIG. 7A presents a first example of graphic representation in the form of a bar chart, of a multiparameter graphic indicator produced by a method according to the invention, said indicator describing a plurality of estimated quantities of interest relative to a plurality of respective standard quantities of interest on normed axes, in a patient suffering from acute SAR;

FIG. 7B presents a second example of graphic representation, in the form of a radar chart, of a multiparameter graphic indicator produced by a method according to the invention, said indicator showing a plurality of estimated quantities of interest relative to a plurality of respective standard quantities of interest on normed axes, in a patient suffering from acute SAR;

FIG. 8 presents a first example of graphic representation in the form of a bar chart, of a multiparameter graphic indicator produced by a method according to the invention, said indicator describing a plurality of estimated quantities of interest relative to a plurality of respective standard quantities of interest on normed axes, in a patient suffering from acute SAR;

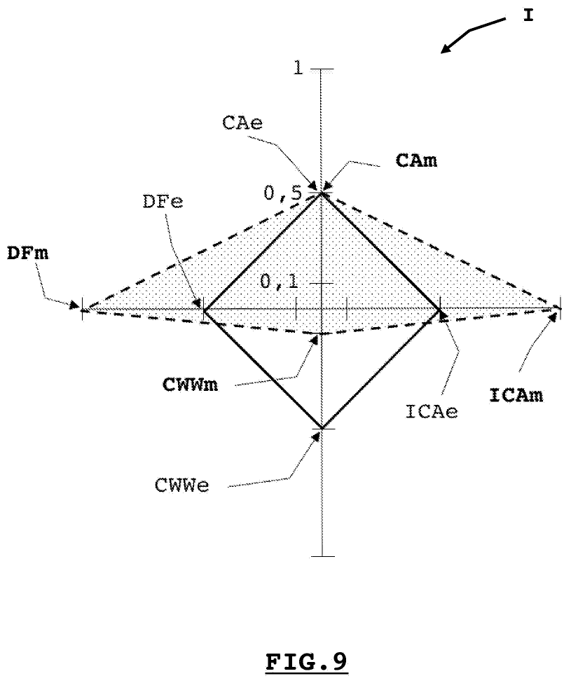

FIG. 9 presents a second example of graphic representation, in the form of a radar chart, of a multiparameter graphic indicator produced by a method according to the invention, said indicator showing a plurality of estimated quantities of interest relative to a plurality of respective standard quantities of interest on normed axes, in a patient suffering from acute SAR;

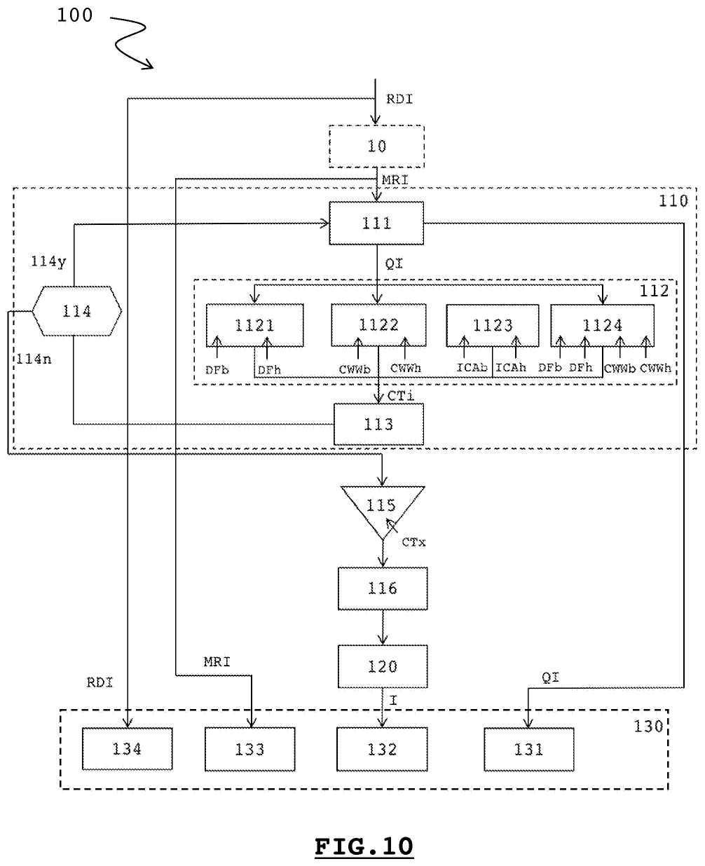

FIG. 10 presents a simplified flow chart showing a non-limitative example of a method for producing a multiparameter graphic indicator relative to a tissue of a human or animal organ according to the invention.

FIG. 1 describes a first digital representation RDI of a histological section from a lung of a patient suffering for example from a remodelling of the small airways or SAR. Said affected patient is in this case a mouse. Such a first representation RDI generally originates from a process of scanning a histological section. A digitized histological section with ×20 enlargement delivers said first digital representation in a matrix form with approximately two hundred million pixels, i.e. according to the example in FIG. 1, a representation in the form of an array of fifteen thousand rows on the same number of columns, each element of said array encoding a triplet of integer values comprised between zero and two hundred and fifty-five, according to the RGB (red, green, blue) colour model. Such a computerized colour model is the most used by the available material. In general, computer screens reconstitute a colour by additive synthesis from three primary colours, a red, a green and a blue, forming on the screen a mosaic that is generally too small to be distinguished by humans. The RGB model gives a value for each of these primary colours. Such a value is generally coded on one byte and thus belongs to an integer value interval comprised between zero and two hundred and fifty-five.

The RDI representation presents, in a distinguishable manner in the centre of said representation, the lobe L of a lung. Moreover, such an organ comprises numerous substantially tubular components Ci, wherein the inner walls respectively form lumina. Following the sectioning performed at the level of the pulmonary lobe, only sections of said tubular components Ci are visible, in two dimensions. The latter can be described, in particular with reference to FIG. 3, as annular structures, wherein the ring is a section of the wall of the component Ci, encircling a hole associated with the lumen. Such tubular components Ci consist mainly of vasculature, bronchi, bronchioli or alveoli being able to form alveolar sacs. The remainder of the tissue P of said lobe is hereinafter called “parenchyma”.

When a patient is suffering for example from a chronic obstructive respiratory disease, the lesion of the lung is revealed by changes in the morphology of some of the tubular components forming the lung, in particular by a remodelling of the airways, more particularly of the bronchioli in the case of SAR. The diameters of the bronchioli are generally comprised, within the taxonomy of the vertebrates, between one hundred microns and one thousand microns. Thus in a patient in the early stages of airway disease, SAR involves desquamation of the bronchial epithelium, more commonly called “bronchial wall” characterized on a digital representation of a histological section of a lung, by a ring representing a section of such a bronchial wall. Such desquamation generally results in a reduction of the area of the bronchioli and of said bronchial wall, characterized on a digital representation of a histological section of a lung by a reduction of the area of the ring and possibly of the area encircled by said ring, i.e. the area of the section of a lumen of said tubular component. At a later stage, a more severe disorder results in a remodelling of such bronchioli and a modification of the metabolism thereof, evidenced by chronic inflammation of the bronchial wall leading to changes in the morphology of said bronchioli. This then results in an increase of the mean lumen and of the area of said remodelled bronchioli, as well as an increase in the mean thickness of the bronchial epithelium thereof. Such a disorder is characterized, on a digital representation of a histological section of a lung, by an increase in the area of the annular structure, i.e. the area of the section of said tubular component, and more particularly the area encircled by said ring, i.e. the area of the transverse section of a lumen of said tubular component. FIG. 1 thus describes a first digital representation RDI of a lobe L of a lung OG of a patient suffering from SAR.

Said first digital representation RDI is, according to the state of the art, difficult to use so that an investigator can determine the presence of morphometric modifications resulting from possible remodelling of the small airways, characterizing a pathology such as SAR. In fact, said morphometric modifications being of the order of a micron, it is very difficult for an investigator to observe them based on a histological section. Thus, according to the state of the art, the aforementioned techniques are preferred for characterizing, in patients, a disorder of the small airways.

In order to produce an objective, automated and almost real-time aid to such investigators, the invention envisages focusing on the morphometric properties of the components located in a histological section of a lung by using a digital representation, obtained by scanning said histological section. So as to facilitate understanding of the method for estimating a quantity of interest according to the invention, a single digital representation of a histological section of a patient presenting a disorder of the small airways is shown, the differences from a digital representation of a histological section of a healthy patient being imperceptible to the human eye.

Moreover, in order to facilitate finding components of interest, a method according to the invention can comprise a prior step 10 consisting of processing to binarize said first digital representation RDI and to produce a second digital representation MRI. Such a second digital representation MRI can be produced by any known type of digital processing intended to binarize a digital representation RDI in colour(s), such as the digital representation RDI. Such a digital representation or image is revealed in the form of an array comprising one and the same number of elements or pixels as the first digital representation RDI of a histological section wherein it originates, such as the first digital representation RDI mentioned above with reference to FIG. 1. Said second digital representation MRI is called binary because each of the elements thereof MRI(i,j), denoted by two indices i and j respectively determining the row and the column of said element or pixel in the array MRI, comprises an integer value chosen from two predetermined values respectively signifying that the pixel RDI(i,j), i.e. of one and the same column j and one and the same row i in a first digital representation RDI, denotes a portion of the lobe L or not.

By way of non-limitative example, FIG. 2 shows an example second binary representation MRI, the second binary digital representation MRI in this case originating from the first digital representation RDI shown with reference to FIG. 1. According to this example, an element MRI(i,j) of the array MRI adopts the value zero if the associated pixel RDI(i,j), i.e. denoted by the row i and the column j in the first digital representation RDI, does not correspond to a pixel of the lobe L, i.e. said pixel describes either a lumen formed by the transverse section of a tubular component, or the exterior of the lobe L of the lung. In the alternative, such an element MRI(i,j) adopts the value two hundred and fifty-five. In this way, such a second binary digital representation MRI can be displayed in black and white on a computer screen. The invention should not be limited to the use of said values zero and two hundred and fifty-five. In a variant, other predetermined values could have been chosen than said values zero and two hundred and fifty-five to characterize the absence of interest or the interest of such a pixel.

Advantageously but non-limitatively, to facilitate the estimation of quantities of interest relating to annular components present in a digital representation of a histological section of a tissue of a human or animal organ, and ultimately the production of a multiparameter graphic indicator, processing 10 for producing a binary representation MRI of a digital representation RDI can be implemented before implementation of a method for producing a multiparameter indicator according to the invention, such as a method 100, wherein a non-limitative example embodiment is in particular described with reference to FIG. 10. For the sake of brevity and simplicity, reference will be made to morphology of the component Ci instead of the morphology of the section of annular shape of a tubular component, as described with reference to FIG. 3. Thus, by application of the histological section, a tubular component of interest Ci is revealed in a digital representation RDI or MRI by a two-dimensional annular structure. Said processing 10 intended to binarize a digital representation can, in a variant, constitute a first step of said method 100 mentioned above. A non-limitative example of processing 10 intended to binarize such a first digital representation RDI, can comprise a first step for producing a first intermediate digital representation in greyscale, not shown in the figures for the sake of simplicity. Said intermediate digital representation comprises one and the same number of elements or of pixels as the first digital representation RDI. Such a first step can consist of the implementation of any known technique for converting, for each pixel of the representation RDI, the triplet of values representing the primary colour scales to an integer value representing a colour value or luminous intensity associated with a thus-produced representation pixel. Said first step can also consist of the application, on the thus-produced digital representation, of a median or bilateral filter to suppress certain anomalies.

A second step of such processing 10 intended to binarize a digital representation according to the invention can consist of implementing automatic thresholding of the pixels of the (first) intermediate digital representation, so as to distinguish the pixels describing all or part of a lumen formed by the transverse section of a tubular component or the exterior of the lobe L of the lung. The pixels associated with a lumen or with the exterior of the lobe, adopt the value zero, thus appearing in black in FIG. 2. The other pixels adopt the value two hundred and fifty-five and appear in white. They are associated with parenchymal tissue or with certain tubular components such as vasculature, alveoli, alveolar sacs, bronchi or bronchioli. This second step thus produces a second binary digital representation MRI.

Such processing 10 intended to binarize a digital representation can, in addition, produce a third binary digital representation, which will be called “Lobe mask”, having the same dimensions as the second binary digital representation MRI, wherein each element comprises a first value specifying that an associated pixel within a digital representation RDI or MRI belongs to the lobe or is exterior thereto. In fact, taking account in particular in the second binary digital representation MRI of pixels associated with the background AP of the lobe L can affect the relevance of the quantities of interest produced and ultimately the relevance of a multiparameter graphic indicator produced by a method according to the invention. To this end, in a non-limitative example of a method according to the invention, a pixel from the second binary digital representation MRI will only be taken into consideration if, and only if, the associated pixel in said lobe mask, i.e. having the same row and column index, comprises a value characterizing a pixel belonging to the examined lobe L. Such a third digital representation, not shown in the Figures for the sake of simplicity, can be produced in addition to the second step of processing 10 intended to binarize a digital representation, by finding the greatest contour by implementing, for example, a “flood-fill” algorithm, also known as “spread-fill algorithm”.

FIG. 10 shows a non-limitative example embodiment of a method 100 for producing a multiparameter graphic indicator I relative to remodelling of the epithelium of a human or animal organ, based on a digital representation of a histological section of said organ according to the invention, whether such a representation is in the form of a colour RDI or binary MRI digital representation.

Such a method 100 and/or such prior processing 10, intended to binarize a digital representation RDI and produce a digital representation MRI can be arranged to be transcribed into a computer program, wherein the program instructions can be installed in a program memory of an electronic object, in the form for example of a computer having sufficient processing power and/or suitable for the analysis of digital representations or images of concomitant sizes, taking account of the accuracy necessary for analysis of a pulmonary lobe L.

Said program instructions are thus arranged to cause the implementation of said method 100 and prior processing 10 intended to binarize a digital representation RDI by the processing unit of such an electronic object. Within the meaning of the present document, by “processing unit” is meant one or more microcontrollers or microprocessors cooperating with a program memory hosting the computer program according to the invention. Such a processing unit can moreover be arranged to cooperate with a data memory to host, i.e. store, the digital representations produced by the implementation of a method 100 to produce a multiparameter graphic indicator I relative to a remodelling of the bronchial epithelium according to the invention, and/or all other data necessary for the implementation thereof.

Such a processing unit can also be arranged to cooperate with an output human-machine or peripheral interface, such as a computer screen, a printer or any other interface for delivering the content of said multiparameter graphic indicator I to a human being, so as to be perceived by means of one of the senses.

With reference to FIG. 10, such a method 100 for producing a multiparameter graphic indicator I can comprise a sequence 110, optionally iterative, of steps intended to estimate, from a first digital representation RDI and/or MDI of a histological section of a tissue of a human or animal organ, one or more quantities of interest QI relative to the respective morphologies of the components Ci present in said histological section.

Said iterative sequence 110 of a method for producing a multiparameter graphic indicator I according to the invention comprises a step 111 for estimating one or more quantities of interest QI. Implementation of step 111 can advantageously comprise a succession of sub-steps, not shown in FIG. 10, consisting of “finding” from a digital representation RDI and/or MDI of a histological section, a first lumen described by the section of a substantially annular component Ci. By the application of a technique such as described by Satochi Suzuki et al. “Topological structural analysis of digitized binary images by border following”, Computer Vision, Graphics, and Image processing, 1985 or any other equivalent technique, step 111 consists of determining, from digital representations RDI and/or MRI, topologies of components Ci present in said digital representation RDI and/or MRI. It is then possible to determine the contour of an area associated with a lumen of a component Ci identified beforehand, the latter resembling on a binary digital representation MRI a set of contiguous pixels describing for example a number of values equal to zero. The step 111 thus consists of delimiting the inner contour of said lumen i.e. consequently, that of the inner wall of said identified component. The result of the application of such a technique is revealed by a first polyline wherein the indices, i.e. the columns and rows of the pixels constituting the characteristic points therein, describe said contour of the inner wall, encircling the lumen identified beforehand of a tubular component Ci. Step 111 currently consists of determining, from such a first polyline, the contour of the outer wall of the component Ci identified beforehand. By way of non-limitative example, such a step 111 for estimating one or more quantities of interest of such a method 100 to produce a multiparameter graphic indicator I can implement a technique, such as that called “morphological dilation of the inner contour” described in the work by Jean Serra, Image Analysis and Mathematical Morphology, 1982, or any other equivalent technique. Utilization of such a technique thus produces a second polyline wherein the indices of the pixels, constituting the characteristic points therein, make it possible, together with said first polyline produced beforehand, to estimate the morphology of the section of an annular-shaped component, defined by the first and second thus-determined polylines. From said first and second polylines, the step 111 of a method 100 for producing a multiparameter graphic indicator I according to the invention currently consists of estimating a quantity of interest QI relating to said morphology of said component Ci wherein the lumen was identified beforehand.

A component of interest Ci thus has, in two dimensions, a substantially annular shape as described in an example with reference to FIG. 3, advantageously but non-limitatively for a type CTi of component Ci, in this case a bronchiolus. Said bronchiolus, and more generally a type of component Ci, comprises an outer contour CWOCi, an inner contour CWICi encircling a lumen CLi, and a wall CWi, wherein the corresponding areas are denoted by a blunt arrow. As specified above, different quantities of interest QI can be estimated in step 111 of a method according to the invention. As described with reference to FIG. 3, such quantities of interest may consist of:
the Feret diameter DFi of a component Ci identified beforehand. Within the meaning of the invention and throughout the document, by “Feret diameter” is meant the greatest distance between two tangents to the second polyline describing the apparent outer contour CWOCi of the wall CWi of said component Ci, said tangents being parallel to one another and perpendicular to a given direction vector. By varying the polar angle of said vector from 0 to 2π during the implementation of step 111, a set of diameters can thus be estimated, wherein the largest corresponds to the Feret diameter DFi retained for characterizing said component Ci;
The mean distance CWWi, which will be called mean thickness CWWi for the purposes of simplification, of the wall CWi of a component Ci identified beforehand. Within the meaning of the invention and throughout the document, by “mean thickness CWWi” is meant the ratio between the sum of the estimated distances separating the outer contour CWOCi from the inner contour CWICi of said component Ci identified beforehand and the number of estimated distances. In the context of pathologies affecting the airways, such as SAR, the wall CWi of certain components Ci found in the lungs may suffer total or partial desquamation. Thus, estimation of such a mean distance CWWi of a component Ci is particularly relevant, since it makes it possible to describe the significance and the extent of such a desquamation of the wall CWi of an annular component Ci and consequently the desquamation of the tubular component Ci, wherein the annular component Ci describes a section thereof;
the area ICAi described by the inner contour CWICi of a component Ci, revealing the lumen CLi thereof. Within the meaning of the invention and throughout the document, by “area ICAi” is meant the surface area of a section describing the lumen CLi of a component Ci, said surface area being delimited by the first polyline of said component Ci;
the area OCAi described by the outer contour CWOCi of a component Ci, revealing the total area covered thereby. Within the meaning of the invention and throughout the document, by “area OCAi” is meant the surface area describing the lumen CLi of a component Ci, said surface area being delimited by the first polyline of said component Ci, as well as the surface area described by the wall of said component Ci, said surface area describing the wall being delimited by the first polyline and the second polyline of said component Ci;
the area CAi described by the wall CWi of a component Ci, revealing the area covered thereby. Within the meaning of the invention and throughout the document, by “area CAi” is meant the surface area describing the wall CWi, said wall CWi being delimited by the first and second polylines of said component Ci.

By way of advantageous but non-limitative example, implementation of step 111 of a method 100 for producing a multiparameter graphic indicator I according to the invention makes it possible, from first and second polylines produced beforehand, respectively associated with the inner contour CWICi and the outer contour CWOCi, to estimate one or more quantities of interest QI, such as, the Feret diameter DFi of a component Ci. A non-limitative example of such a step 111 for estimating such a diameter consists of adding the pixels of known dimensions, in order to deduce therefrom a corresponding distance characterizing said diameter. The relative distance at each diameter can thus be estimated, and the greatest distance corresponding to the Feret diameter DFi may thus be recorded, during the implementation of a step 113 of a method 100 for producing a multiparameter graphic indicator I according to the invention, in a data structure associated with the component Ci.

Step 111 of a method 100 for producing a multiparameter graphic indicator I according to the invention can, in a variant or in addition, consist of estimating a mean distance CWWi revealing a mean thickness of the wall CWi of a component Ci identified beforehand. An example of such a step 111 for estimating such a mean distance CWWi can consist of estimating, for each pixel of a first and/or a second polyline respectively describing the inner contour CWICi and the apparent outer contour CWOCi of the wall CWi of said component Ci, the smallest distance separating said pixel from said first and/or second polylines. A plurality of estimations of distances describing the thickness of the wall CWi of the corresponding component Ci can thus be estimated, so that by averaging said estimations, the implementation of such a step 111 makes it possible to estimate a mean distance CWWi revealing the mean thickness of the wall CWi of said component Ci. Said mean distance may be recorded during the implementation of a step 113 of a method 100 for producing a multiparameter graphic indicator I according to the invention, in a data structure associated with the component Ci.

Step 111 of a method 100 for producing a multiparameter graphic indicator I according to the invention can, in a variant or in addition, consist of estimating an area ICAi revealing the surface area of the lumen CLi of a component Ci identified beforehand. An example of such a step 111 for estimating such an area ICAi can consist of adding the pixels having known dimensions describing the surface area delimited by said first polyline. Implementation of such a step 111 thus makes it possible to estimate the area ICAi, corresponding to the sum of the areas of the pixels describing the lumen CLi, of the component Ci identified beforehand. Said area ICAi may be recorded during the implementation of a step 113 of a method 100 for producing a multiparameter graphic indicator I according to the invention, in a data structure associated with the component Ci.

Moreover, step 111 of a method 100 for producing a multiparameter graphic indicator I according to the invention can consist of estimating an area OCAi revealing the surface area of a component Ci identified beforehand. An example of such a step 111 for estimating such an area OCAi can consist of adding the pixels having known dimensions describing the area delimited by said second polyline. Implementation of such a step 111 makes it possible to estimate the area OCAi, corresponding to the sum of the areas of the pixels describing the totality of the area of the component Ci identified beforehand. Said area OCAi may be recorded during the implementation of a step 113 of a method 100 for producing a multiparameter graphic indicator I according to the invention, in a data structure associated with the component Ci.

Advantageously, step 111 of a method 100 for producing a multiparameter graphic indicator I according to the invention can consist of estimating an area CAi revealing the surface area of the wall CWi of a component Ci identified beforehand. An example of such a step 111 for estimating such an area CAi can consist of subtracting the area ICAi, described by the inner contour CWICi of a component Ci, from the area OCAi, described by the outer contour CWOCi of the same component Ci, both estimated beforehand. Said area CAi may be recorded during the implementation of a step 113 of a method 100 for producing a multiparameter graphic indicator I according to the invention, in a data structure associated with the component Ci.

As mentioned above, the sequence 110 of a method 100 for producing a multiparameter graphic indicator I according to the invention can also comprise a step 113 for registering in the data memory a data structure associated with each identified component Ci, wherein the morphology was characterized during a step 111 of said method 100. Said data structure can advantageously comprise a field for storing the value of each corresponding estimated quantity of interest QI with reference to said component Ci, such as, for example, the Feret diameter DFi of said component Ci, a mean thickness CWWi of the wall CWi of said component Ci, the area ICAi describing the lumen CLi of said component Ci, the area OCAi describing the total surface area of said component Ci, and the area CAi describing the total surface area of the wall CWi of said component Ci.

Advantageously but non-limitatively, as detailed above, said sequence can be implemented iteratively for one or more contours of one or more lumina CLi characteristic respectively of one or more identified components Ci. To this end, according to a non-limitative embodiment of a method 100 for producing a multiparameter graphic indicator I according to the invention described with reference to FIG. 10, such a sequence can also comprise a test step 114, so that when there is no longer any other contour encircling a lumen CLi characteristic of a still unidentified component Ci, situation illustrated by the link 114n in FIG. 10, estimation of one or more quantities of interest by iteration, as shown by the link 114y between step 113 and step 111, comes to an end and the data relating to said quantities of interest are ready to be used by the step 120 of the method 100 shown by way of non-limitative example in FIG. 10.

In order to provide an investigator with an aid to diagnosis of a pulmonary pathology or in the analysis of the therapeutic efficacy of a molecule, a method 100 for producing a multiparameter graphic indicator I according to the invention can comprise a step 112, prior to step 113 for registering in the data memory a data structure associated with a component Ci for characterizing a type CTi of component Ci from the value of one of the quantities of interest QI estimated during step 111, present in a histological section of a lung OG, such as by way of non-limitative example, types of components distinguishing alveoli and/or alveolar sacs, bronchioli, bronchi and vessels. In fact, as said types of tubular components mentioned above have annular bodies of very different morphologies, it may then be advantageous to characterize the type of each identified component Ci in order to be able to determine as accurately or precisely as possible if said components Ci have modifications of their morphology, as is often the case in the context of pathologies affecting the pulmonary tract.

By way of non-limitative example, according to an embodiment of a method 100 for producing a multiparameter graphic indicator I according to the invention, a step 112 for characterizing a type CTi of component Ci can consist of comparing, in a sub-step 1121, the value of the Feret diameter DFi of a component Ci estimated during the implementation of step 111, to a high-diameter threshold DFh and/or a low-diameter threshold DFb, said thresholds DFh and DFb advantageously being parameterized beforehand. Thus, in order to characterize a component Ci of the bronchiolus type, said sub-step 1121 may consist of assigning a predetermined value, for example the value “1” to a dedicated field in the data structure associated with said component Ci, if the Feret diameter DFi thereof is greater than a low-diameter threshold DFb of one hundred micrometres and less than a high-diameter threshold DFh of one thousand micrometres. In fact, the types CTi of components Ci observed in a histological section of a lung OG generally have a diameter substantially comprised between ten micrometres and one thousand micrometres. Use of the Feret diameter proves particularly advantageous, since such a use thus facilitates the classification of the different types CTi of components Ci and allows an investigator to only take into consideration one or more types of components of interest from the different types CTi of identified components Ci, in order subsequently to produce a multiparameter graphic indicator I providing a valuable aid in the establishment of a diagnosis of a pathology affecting the respiratory tract.

In a variant or in addition, still with reference to FIG. 10, a step 112 for characterizing a type CTi of a component Ci of a method 100 for producing a multiparameter graphic indicator I according to the invention can consist of comparing, in a sub-step 1122, the value of the mean thickness CWWi of a component Ci estimated during the implementation of step 111, to a high mean-thickness threshold CWWh and/or a low mean-thickness threshold CWWb, said thresholds CWWh and CWWb advantageously being capable of being parameterized beforehand. Thus, in order to characterize a component Ci of the bronchiolus type, said sub-step 1122 may consist of allocating said predetermined value “1” in a dedicated field in the data structure associated with said component Ci, if the mean thickness CWWi thereof is greater than a high-thickness threshold CWWh of ten micrometres. In fact, the types CTi of components Ci observed in a histological section of a lung OG generally have a mean thickness CWWi greater than 10 micrometres. Use of the mean thickness CWWi of the wall CWi can facilitate the classification of the different types CTi of components Ci and allow an investigator to only take into consideration the type or types CTi of components Ci of interest, in order subsequently to produce a multiparameter graphic indicator I providing a valuable aid in the establishment of a diagnosis of a pathology affecting the respiratory tract.

Advantageously, in a variant or in addition, according to FIG. 10, a step 112 for characterizing a type CTi of component Ci of a method 100 for producing a multiparameter graphic indicator I according to the invention can consist of comparing, in a sub-step 1123, the value of the area ICAi described by the inner contour CWICi of a component Ci estimated during the implementation of step 111, to a high-area threshold ICAh and/or a low-area threshold ICAb, said thresholds ICAh and ICAb advantageously being capable of being parameterized beforehand. Thus, in order to characterize a component Ci of the bronchiolus type, said sub-step 1123 may consist of assigning the predetermined value “1” in a dedicated field to the type of component in the data structure associated with said component Ci, if the area ICAi thereof is less than a low-area threshold ICAb of twelve thousand square micrometres and greater than a high-area threshold ICAh of fifteen thousand square micrometres. In fact, the types CTi of components Ci observed in a histological section of a lung OG, more particularly the bronchioli, generally have an area ICAi substantially equal to thirteen thousand square micrometres. Use of the area ICAi thus facilitates the classification of the different types CTi of components Ci and/or differentiation of the different stages of the disorder such as acute and chronic stages, in the context of a pathology such as SAR, and allows an investigator to only take into consideration the type or types CTi of the components Ci judged of interest, in order subsequently to produce a multiparameter graphic indicator I providing a valuable aid in the establishment of a diagnosis of a pathology affecting the respiratory tract.

Three examples of characterization of types CTi of components Ci have been described above, as a function in particular of an estimated quantity of interest. However, in certain cases, use of a single quantity of interest is not sufficient to distinguish certain types CTi of components, for example the alveoli and bronchioli. In order to overcome this drawback, the invention provides that the step 112 for characterizing a type CTi of component Ci of a method 100 for producing a multiparameter graphic indicator I according to the invention may be arranged to use different quantities of interest together. According to an embodiment of such a method 100 described with reference to FIG. 10, said step 112 can firstly consist of comparing, in a sub-step 1124, the value of the Feret diameter DFi of a component Ci estimated during the implementation of step 111 to a high-diameter threshold DFh and/or a low-diameter threshold DFb, said thresholds DFh and DFb advantageously being capable of being parameterized beforehand. If said value of the Feret diameter DFi of a component Ci is comprised between said thresholds DFb and DFh, said sub-step 1124 can then consist of comparing the value of the mean thickness CWWi of said component Ci estimated during implementation of step 111 to a high mean-thickness threshold CWWh of ten micrometres, advantageously to a high mean-thickness threshold CWWh of five micrometres. In fact, in the context of airways affected by a pathology such as SAR, it is known that the bronchioli suffer a remodelling, being characterized in particular by an increase of the area of the bronchial wall and/or the area of the lumen CLi of said bronchioli. In a highly inventive manner, a method 100 for producing a multiparameter graphic indicator I according to the invention intends that said sub-step 1124 is arranged, firstly, to compare the value of the Feret diameter DFi of a component Ci to a low-diameter threshold DFb of one hundred micrometres and a high-diameter threshold DFh of one thousand micrometres. In order to characterize the type of different components Ci wherein the Feret diameter DFi is comprised within such an interval, from one hundred micrometres to one thousand micrometres, in this case the components Ci of the bronchiolus, alveolus and/or alveolar sac type, said sub-step 1124 can consist, secondly, of comparing the value CWWi of said component Ci estimated during implementation of step 111, to a high mean-thickness threshold CWWh of five micrometres. Thus, if such a mean thickness CWWi is greater than said thickness threshold CWWh, then said sub-step 1124 may consist of assigning said predetermined value “1” in a dedicated field in the data structure associated with said component Ci. Conversely, if such a mean thickness CWWi is less than said thickness threshold CWWh, then said sub-step 1124 may consist of assigning said predetermined value “2” in a dedicated field in the data structure associated with said component Ci. Such a predetermined value “1” may, by way of advantageous but non-limitative example, characterize a component Ci of the bronchiolus type. Implementation of such a sub-step 1124, using a plurality of quantities of interest together, can thus facilitate classification of the different types CTi of components Ci wherein the morphologies are similar, such as for example the components Ci of the bronchiolus type and of the alveolus and/or alveolar sac type. In fact, it is advantageous in particular to characterize such bronchioli in order to be able to estimate the degree of severity with which a patient is suffering from a pathology such as SAR, relating to the remodelling of the components Ci of the bronchiolus type.

Thus, according to a preferred but non-limitative embodiment of a method 100 for producing a multiparameter graphic indicator I according to the invention, the step 113 for registering, in the data memory, a data structure associated with each identified component Ci can moreover consist of registering a predetermined value in a dedicated field of said data structure to characterize, based on one or more quantities of interest, a particular type CTi of a component Ci, such as for example the bronchiolus type.

Furthermore, in addition, according to FIG. 10, a method 100 for producing a multiparameter graphic indicator I according to the invention can comprise a step 115 for filtering the data estimated beforehand and recorded in the data memory, making it possible to take account of only data relating to one or more determined types CTx from the set of values of types CTi of identified components Ci, in order subsequently to finally produce a multiparameter graphic indicator I relating to said data. By way of non-limitative example, such a step 115 can consist of reading, in the data structure associated with a component Ci, the value present in the field characterizing the type CTi of said component Ci. To this end. said step 115 can advantageously be parameterized such that only the data, more particularly the quantity or quantities of interest estimated beforehand relative to the components Ci of type CTx “bronchiolus” associated with such a type of component Ci comprising, in the field characterizing the type CTi of said component Ci, a predetermined value CTx for example equal to “1”, are used to subsequently produce a multiparameter graphic indicator I, with a view to providing an aid in the diagnosis of a pathology such as, by way of non-limitative example, SAR.

Moreover, in order to compare the values of the quantities of interest estimated beforehand to values of the standard quantities of interest and finally to facilitate the production of a multiparameter graphic indicator I, it may be necessary to calculate or estimate a mean of the quantities of interest. Thus, advantageously but non-limitatively, a method 100 for producing a multiparameter graphic indicator I according to the invention can comprise a step 116 for estimating a mean for each set of quantities of interest QI per type CTi of component Ci. Step 116 can thus produce, for a given type CTi of component Ci optionally filtered beforehand during the implementation of step 115, one or more mean quantities of interest QI, from quantities of interest QI estimated respectively for all the components Ci. By “set of quantities of interest QI” is meant all the values estimated beforehand, for example relative to:
the Feret diameter DFi of the outer contour CWOCi of the wall CWi for a given type CTi of component Ci;
the mean distance CWWi separating an outer contour CWOCi from an inner contour CWICi for a given type CTi of component Ci;
the area ICAi described by an inner contour CWICi for a given type CTi of component Ci;
the area OCAi described by an outer contour CWOCi for a given type CTi of component Ci;
the area CAi of the wall CWi defined by subtracting the area ICAi described by an inner contour CWICi from the area OCAi described by an outer contour CWOCi for a given type CTi of component Ci.

By way of non-limitative example, to produce a multiparameter graphic indicator I making it possible to characterize a pathology such as SAR, step 116 of a method 100 for producing a multiparameter graphic indicator I according to the invention can consist of estimating, for a set of components Ci of the bronchiolus type:
the mean Feret diameter DFm corresponding to the ratio between the sum of the Feret diameters DFi of the components Ci of the bronchiolus type and the number of components Ci wherein a Feret diameter DFi was estimated beforehand;
the mean thickness CWWm corresponding to the ratio between the sum of the mean thicknesses CWWi of the components Ci of the bronchiolus type and the number of said components Ci wherein said thickness CWWi was estimated beforehand;
the mean area ICAm of the lumina CLi corresponding to the ratio between the sum of the estimated areas ICAi of the components Ci of the bronchiolus type and the number of components Ci wherein the area ICAi was estimated beforehand;
the mean area OCAm corresponding to the ratio between the sum of the areas OCAi of the components Ci of the bronchiolus type and the number of components wherein the area OCAi was estimated beforehand;
the mean area CAm corresponding to the ratio between the sum of the areas CAi of the components Ci of the bronchiolus type and the number of components wherein the area CAi was estimated beforehand.

Advantageously, a method 100 for producing a multiparameter graphic indicator I according to the invention can comprise a step 120 consisting of producing one or more graphic representations, intended to transcribe one or more quantities of interest QI such as, by way of non-limitative example, the quantities of interest DFi, CWWi, ICAi, OCAi, CAi estimated beforehand, in a graphic form. Said method 100 can thus comprise a step 130 for causing the joint graphic outputting of said graphic representations produced beforehand in step 120, and deliver a multiparameter graphic indicator I.

Preferentially but non-limitatively, a step 120 of a method 100 for producing a multiparameter graphic indicator I according to the invention can consist of producing a graphic representation, for a type CTi of component Ci given and/or selected beforehand by means of a human-machine input interface, a quantity of interest QI relative to a standard quantity of interest. By “standard quantity of interest” is meant a reference quantity of interest that can be optionally parameterized, associated with each estimated quantity of interest QI. According to the examples described above, such a standard quantity of interest can consist of, non-limitatively:
the mean Feret diameter DFe corresponding to the ratio between the sum of the Feret diameters DFi of the components Ci of the bronchiolus type and the number of components Ci wherein a Feret diameter DFi was estimated beforehand in a healthy patient;
the mean thickness CWWe corresponding to the ratio between the sum of the mean thicknesses CWWi of the components Ci of the bronchiolus type and the number of said components Ci wherein said thickness CWWi was estimated beforehand in a healthy patient;
the mean area ICAe of the lumina CLi corresponding to the ratio between the sum of the estimated areas ICAi of the components Ci of the bronchiolus type and the number of components Ci wherein the area ICAi was estimated beforehand in a healthy patient;
the mean area OCAe corresponding to the ratio between the sum of the areas OCAi of the components Ci of the bronchiolus type and the number of components wherein the area OCAi was estimated beforehand in a healthy patient;
the mean area CAe corresponding to the ratio between the sum of the areas CAi of the components Ci of the bronchiolus type and the number of components wherein the area CAi was estimated beforehand in a healthy patient.

A healthy patient is defined as a patient having no pathology affecting the airways, such as, by way of non-limitative example, SAR. In this case, the graphic representations of the quantities of interest QI of a type CTi of component Ci produced during the step 120 for producing a graphic representation and/or the step 130 for producing a multiparameter graphic indicator I can be expressed as a relative or normalized value, thus allowing said quantities of interest to be expressed according to a common scale and consequently comparable together, such quantities of interest being preferentially but non-limitatively expressed as a percentage of the corresponding standard quantities of interest.

Preferentially, according to a first example of graphic representation described with reference to FIG. 4A, such a step 120 can consist of producing a graphic representation in the form of a bar of a bar chart representing, for example, one of the quantities of interest QI estimated beforehand, wherein the height or the length, according to whether the bar is vertical or horizontal respectively, describes the relative value of said estimated quantity of interest with respect to said standard quantity of interest. Said graphic representation can moreover encode one or more data items determined for characterizing, during the display of said graphic representation via an output human-machine interface during step 130, a texture, a pattern or a particular contour, or any other data allowing an effect on the graphic rendering. Advantageously, in a variant, according to a second example of graphic representation described with reference to FIG. 4B, a step 120 of a method 100 according to the invention can consist of producing a graphic representation in the form of a point on a radar chart representing, for example, one of the quantities of interest QI estimated beforehand, wherein the position on said axis describes the relative value of said estimated quantity of interest with respect to said standard quantity of interest. Thus, according to the first and second examples of graphic representations described respectively with reference to FIGS. 4A and 4B, the estimated mean quantities of interest DFm, CAm, CWWm, ICAm for a type CTi of component Ci, of a digital representation of a histological section of an organ of a patient, can be compared together and respectively to the standard quantities of interest DFe, CAe, CWWe, ICAe corresponding to a healthy patient.

According to a preferred, but non-limitative, embodiment of a method 100 for producing a multiparameter graphic indicator I according to the invention, the production of the graphic representations in step 120 of said method 100 will be described for a type of component Ci of interest in the form of a bronchiolus, with a view to producing a multiparameter graphic indicator I. Such a multiparameter graphic indicator I is described in particular with reference to FIGS. 4A, 4B, 5 and 6, after implementation of the step 130 for producing the joint graphic rendering of the graphic representations of the quantities of interest, such as, for example, the mean quantities of interest DFm, CWWm, ICAm, CAm, relative to the mean quantities of interest DFe, CWWe, ICAe, CAe in a healthy patient. Advantageously, said quantities of interest DFm, CWWm, ICAm and CAm can be expressed respectively as a percentage or normalized with respect to a standard quantity of interest, the numerical value of the percentage being juxtaposed with the digital representation in question. In a variant or in addition, a grid symbolized, for example, by axes “50% CTL” and “100% CTL” described as “normalized bars”, as shown with reference to FIG. 4A, or by lines graduated in twenties, described as “normed axes” as shown with reference to FIG. 4B, may be superposed on the graphic representations during the step 130. An investigator can thus instantly observe whether the quantities of interest DFm, CWWm, ICAm and CAm characterizing the mean morphology of the components of the bronchiolus type present in a digital representation of a histological section of a lung are different or not from the standard quantities of interest observed in a healthy patient.

FIG. 4A shows a first, non-limitative, example of graphic representation, in the form of a bar chart, of a multiparameter graphic indicator produced by a method according to the invention, said indicator describing a plurality of estimated quantities of interest relative to a plurality of respective standard quantities of interest on normed axes, in a patient suffering from acute SAR. According to this first example, according to FIG. 4A, such a step 120 can consist of expressing a first quantity of interest DFm, in the form of a bar, with respect to a standard quantity of interest DFe, symbolized by the axis “100% CTL” of the grid. Thus, the graphic representation DFm encodes a numerical value of seventy-seven percent, meaning that the mean Feret diameter DFm in the patient examined is twenty-three percent less with respect to the mean Feret diameter in a healthy patient. Similarly, and independently of the graphic representation of the first quantity of interest DFm, the step 120 can advantageously consist of expressing a second quantity of interest CWWm, in the form of a bar, with respect to a standard quantity of interest CWWe, symbolized by the axis “100% CTL” of the grid. Thus, the graphic representation CWWm encodes a numerical value of seventy-nine percent, meaning that the mean thickness CWWm in the patient examined is twenty-one percent less with respect to the mean thickness in a healthy patient. Similarly, and independently of the respective graphic representations of the first and second quantities of interest DFm and CWWm, the step 120 can advantageously consist of expressing a third quantity of interest ICAm, in the form of a bar, with respect to a standard quantity of interest ICAe, symbolized by the axis “100% CTL” of the grid. Thus, the graphic representation ICAm encodes a numerical value of eighty-seven percent, meaning that the mean area ICAm of the lumina in the patient examined is thirteen percent less with respect to the mean area of the lumina in a healthy patient. Similarly, and independently of the respective graphic representations of the first, second and third quantities of interest DFm, CWWm and ICAm, the step 120 can advantageously consist of expressing a fourth quantity of interest CAm, in the form of a bar, with respect to a standard quantity of interest CAe, symbolized by the axis “100% CTL” of the grid. Thus, the graphic representation CAm encodes a numerical value of seventy percent, meaning that the mean area CAm of the walls in the patient examined is twenty-one percent less with respect to the mean area of the walls in a healthy patient. It will thus be possible, during the implementation of the step 130, to produce the joint graphic rendering of said thus-produced graphic representations via a human-machine interface output of a system implementing a method 100 according to the invention, in the form, for example, of bar charts, in order to provide an investigator with a multiparameter graphic indicator I, showing the different bars side by side.

Such a first example of joint graphic rendering, in the form of a bar chart, is shown in FIG. 4A for a patient respectively suffering from acute SAR. Thus, said FIG. 4A expresses jointly the respective graphic representations of the first, second, third and fourth quantities of interest DFm, CWWm, ICAm and CAm in the form of a graphic representation of the bar chart type, and expressing the respective values of said quantities of interest DFm, CWWm, ICAm and CAm relative to those of the same quantities of interest DFe, CWWe, ICAe and CAe in a healthy patient. An investigator can thus, by simply displaying the multiparameter graphic indicator I, easily take into account that the mean quantities of interest DFm, CWWm, ICAm and CAm in a patient examined are less than those DFe, CWWe, ICAe and CAe in a healthy patient. Such a multiparameter graphic indicator I is then capable of providing a valuable aid to an investigator or a member of healthcare personnel, in order to diagnose, if necessary, a pathology affecting the pulmonary tract, such as SAR for example. In fact, a multiparameter graphic indicator I, as shown with reference to FIG. 4A, provides relevant information relating to the general morphology of all the components of the bronchiolus type present in a histological section of a lung of a patient suffering from acute SAR. Such a disorder generally results in a more or less heterogeneous desquamation of the bronchial epithelium, i.e. a reduction of the mean thickness CWWi of the wall CWi of the bronchioli. This desquamation of the bronchial wall consequently leads to a reduction of the total area CAi of the bronchiolus, possibly the area ICAi of the lumen CLi and of the Feret diameter DFi.

In a variant or in addition, in order to reinforce the visual discrimination of a multiparameter graphic indicator I relating to remodelling of the human or animal bronchial epithelium, the steps 120 and/or 130 of a method for producing such a multiparameter graphic indicator I can, by way of non-limitative example, consist of expressing, then displaying, averaged quantities of interest, in the form of a graphic representation of the radar chart type, also known as a Kiviat diagram, with respect to or relative to these same standard quantities of interest, said standard quantities of interest being indicated as corresponding to 100% relative to a graduated axis. Such an axis can be graduated, by way of non-limitative example, from zero percent of the value of the corresponding standard quantity in a healthy patient. Thus, each quantity of interest QI can be normalized with respect to the corresponding standard quantity of interest, and they can be capable of being displayed after graphic rendering during step 130 in a continuous scale. FIG. 4B shows a second non-limitative example of graphic representation in the form of a radar chart of a multiparameter graphic indicator I produced by a method 100 according to the invention, said indicator describing a plurality of estimated quantities of interest relating to a plurality of respective standard quantities of interest on normed axes, in a patient suffering from acute SAR.

According to this second example, according to FIG. 4B, such a step 120 can consist of expressing a first quantity of interest DFm, in the form of a position on a graduated axis, with respect to a standard quantity of interest DFe, symbolized by the position 100 on the graduated axis. Similarly, and independently of the graphic representation of the first quantity of interest DFm, the step 120 can advantageously consist of expressing a second quantity of interest CWWm, in the form of a position on a graduated axis, with respect to a standard quantity of interest CWWe, symbolized by the position 100 on the graduated axis. Similarly, and independently of the respective graphic representations of the first and second quantities of interest DFm and CWWm, the step 120 can advantageously consist of expressing a third quantity of interest ICAm, in the form of a position on a graduated axis, with respect to a standard quantity of interest ICAe, symbolized by the position 100 on the graduated axis. Similarly, and independently of the respective graphic representations of the first, second and third quantities of interest DFm, CWWm and ICAm, the step 120 can advantageously consist of expressing a fourth quantity of interest CAm, in the form of a position on a graduated axis, with respect to a standard quantity of interest CAe, symbolized by the position 100 on the graduated axis.

It will thus be possible, during step 130, to produce the joint graphic rendering of said thus-produced graphic representations via a human-machine interface output of a system implementing a method 100 according to the invention, such as, by way of non-limitative example, in the form for example of a radar chart, in order to provide an investigator with a multiparameter graphic indicator I. Such a joint graphic rendering can moreover show the materialization of an area defined by the respective different positions of the normalized quantities of interest on the normed axes. Such a first example of joint graphic rendering, in the form of a radar chart, is shown in FIG. 4B for a patient suffering from acute SAR. Thus, said FIG. 4B expresses jointly the graphic representations of the first, second, third and fourth quantities of interest DFm, CWWm, ICAm and CAm in the form of a graphic representation of the radar chart type. The area, describing the joint positions of the quantities of interest DFm, CWWm, ICAm and CAm delimited by a dotted contour, can thus easily indicate the pathological state of the morphology of the components Ci of interest represented on each axis of the diagram with respect to the area, indicated by the solid line contour, describing a typical morphology of such components in a healthy patient. It is thus easier for the investigator to perceive the distance or the deviation between the pathological state of the morphology described by the area delimited with a dotted line, of a patient examined, with respect to a morphology in a healthy patient. In this case, according to the example shown in FIG. 4B, the components Ci of interest of the bronchiolus type, present in a digital representation of a histological section of a lung, have quantities of interest that have decreased with respect to the same standard quantities of interest.

FIG. 5 shows a third, non-limitative, example of graphic representation, in the form of a bar chart, of a multiparameter graphic indicator produced by a method according to the invention, said indicator describing a plurality of estimated quantities of interest relative to a plurality of respective standard quantities of interest on normed axes, in a patient suffering from acute SAR. As in FIGS. 4A and 4B, FIG. 5 expresses jointly respective graphic representations of the first, second, third and fourth quantities of interest DFm, CWWm, ICAm and CAm in the form of a graphic representation of the bar chart type. However, FIG. 5 shows respective graphic representations of the first, second, third, and fourth quantities of interest DFm, CWWm, ICAm and Cam in the form of signed relative deviations, such deviations consisting of positive or negative numerical values calculated from the following mathematical formula for each quantity of interest QI:

$$Em = \frac{QIm - QIe}{QIe} ,$$

wherein Em consists of the value of the estimated mean deviation, QIm consists of the value of the estimated mean quantity of interest in a patient examined, QIe consists of the value of the standard quantity of interest in a healthy patient.

According to this third example, according to FIG. 5, such a step 120 can consist of expressing signed relative deviations between the first, second, third and fourth quantities of interest DFm, CWWm, ICAm and CAm respectively in the form of bars, with respect to a vertical or horizontal zero datum plane described by an axis “100% CTL”. Such deviations can thus represent a relative or normalized reduction or increase of said first, second, third and fourth quantities of interest DFm, CWWm, ICAm and CAm, with respect to respective standard quantities of interest. In this case, for a vertical plane, a reduction of such a quantity of interest is displayed on the left, while an increase of such a quantity of interest is displayed on the right.

It will thus be possible, during the implementation of the step 130, to cause the joint graphic outputting of said thus-produced graphic representations via an output human-machine interface of a system implementing a method 100 according to the invention, in the form, for example, of bar charts, in order to provide an investigator with a multiparameter graphic indicator I, showing the different bars side by side.

FIG. 6 shows a fourth non-limitative example of graphic representation in the form of a radar chart of a multiparameter graphic indicator I produced by a method 100 according to the invention, said indicator describing a plurality of estimated quantities of interest relative to a plurality of respective standard quantities of interest on normed axes, in a patient suffering from acute SAR. As in FIGS. 4A, 4B and 5, FIG. 6 expresses jointly respective graphic representations of the first, second, third and fourth quantities of interest DFm, CWWm, ICAm and CAm in the form of a graphic representation of the radar chart type. However, FIG. 6 shows respective graphic representations of the first, second, third and fourth quantities of interest DFm, CWWm, ICAm and CAm in the form of signed absolute deviations, such deviations consisting of positive or negative numerical values calculated from the following mathematical formula for each quantity of interest QI:

$$E m = QIm - QIe \to \{ \begin{matrix}E m < 0 \to E m = 0.1 \\ E m \approx 0 \to E m = 0. 5 \\ E m > 0 \to E m = 1\end{matrix} ,$$

wherein □□ consists of the value of the estimated mean deviation value, □□m consists of the value of the estimated mean quantity of interest in a patient examined, □□ consists of the value of the standard quantity of interest in a healthy patient. Such a fourth example of graphic representation, in the form of a radar chart, proves particularly advantageous, since such a graphic representation allows an investigator to easily visualize graphically any significant difference between estimated quantities of interest QI and standard quantities of interest QI, even where these are relatively similar.

According to this fourth example, according to FIG. 6, such a step 120 can consist of expressing absolute deviations determined between the first, second, third and fourth mean quantities of interest DFm, CWWm, ICAm and CAm and first, second, third and fourth corresponding respective standard quantities of interest, respectively in the form of positions on graduated axes. Such deviations can thus represent a normalized absolute reduction or increase, in this case according to three predetermined increasing values, “0.1”, “0.5” and “1” of said first, second, third and fourth quantities of interest DFm, CWWm, ICAm and CAm, with respect to respective standard quantities of interest. The invention should not be limited to the predetermined values of the deviations Em. Advantageously, the deviations or said quantities of interest DFm, CWWm, ICAm and CAm can be expressed respectively by a value characterizing a reduction, an equivalence or an increase with respect to a standard quantity of interest, in this case such an equivalence to a standard quantity of interest is shown on each axis of the chart, by the corresponding “0.5” graduation. Thus, the invention provides a threshold of tolerance that can be parameterized or predetermined, for example plus or minus ten percent around the standard value for thus determining such an equivalence between the estimated quantity of interest and the corresponding standard quantity. Similarly, the invention provides that below such a threshold, said quantity of interest is considered to be less, value “0.1”, or greater, value “1” than said standard quantity of interest. In order to visualize graphically a difference considered significant by the investigator, such a fourth example of graphic representation, in the form of a radar chart, can advantageously be arranged to display an increase or a decrease for each estimated quantity of interest QI with respect to a standard quantity of interest if, and only if, said quantity of interest QI is greater or less than a threshold capable of being parameterized.

In this case, according to FIG. 6, a generalized reduction is observed in the first, second, third and fourth mean quantities of interest DFm, CWWm, ICAm and CAm, said deviations with reference to said first, second, third and fourth mean quantities of interest all being equal to “0.1”. It will thus be possible, during step 130, to produce the joint graphic rendering of said thus-produced graphic representations via a human-machine interface output of a system implementing a method 100 according to the invention, such as, by way of non-limitative example, in the form, for example, of a radar chart, in order to provide an investigator with a multiparameter graphic indicator I. The radar chart shown, with reference to FIG. 6, thus directly describes a reduction of the quantities of interest DFm, CWWm, ICAm and Cam, a reduction of such a quantity of interest is displayed on the graduation “0.1” of the corresponding axis, while an increase of such a quantity of interest is displayed on the graduation “1” of the corresponding axis.

Such a joint graphic outputting can moreover show the materialization of an area defined by the different respective positions of the normalized quantities of interest on the normed axes. Such a first example of joint graphic rendering, in the form of a radar chart, is shown in FIG. 4B for a patient suffering from acute SAR. Like FIG. 4B, the area, describing the joint positions of the quantities of interest DFm, CWWm, ICAm and CAm delimited by a dotted contour, can thus easily indicate the pathological state of the morphology of the components Ci of interest represented on each axis of the diagram with respect to the area, indicated by the solid line contour, describing a typical morphology of such components in a healthy patient. According to FIG. 6, the area describing the pathological state is limited or zero with respect to the area describing the typical morphology.

Like FIGS. 4A, 4B, 5 and 6, FIGS. 7A, 7B, 8 and 9 respectively show first, second, third and fourth non-limitative example of graphic representations in the form of bar charts and radar charts of a multiparameter graphic indicator I produced by a method 100 according to the invention, said indicator describing a plurality of estimated quantities of interest relative to a plurality of respective standard quantities of interest on normed axes, in a patient suffering from chronic SAR. Such a multiparameter graphic indicator I is thus particularly suitable for describing the morphology of the bronchioli. In fact, it is known that a pathology, such as SAR, can result in the long term in chronic inflammation of the bronchioli, producing a remodelling thereof. Like FIGS. 4A, 4B, 5 and 6, FIGS. 7A, 7B, 8 and 9 express jointly respective graphic representations of the first, second, third and fourth quantities of interest DFm, CWWm, ICAm and CAm in the form of graphic representations of the bar chart and radar charts type.

One and the same method 100 for producing a multiparameter graphic indicator I is applied to a second histological section of a lung of a patient suffering this time from chronic SAR. Such a pathology can be easily diagnosed by an investigator in light of one or more multiparameter graphic indicators I. To this end, a multiparameter graphic indicator I, as shown in FIG. 7A, provides relevant information relating to the general morphology of all the components of the bronchiolus type present in a histological section of a lung of a patient suffering from chronic SAR. Such a disorder generally results in a more or less significant inflammation of the bronchioli. FIG. 7A thus presents an overall increase of the quantities of interest DFm, ICAm, characterizing an inflammation of said bronchioli as well as a relative “stabilization” of the quantities of interest CAm, CWm, characterizing a regeneration of the bronchial epithelium, each of said quantities of interest being expressed as a percentage of the corresponding standard quantity of interest, i.e. in this case in a healthy patient.

Moreover in a patient suffering from chronic SAR, with reference to FIG. 7B, the investigator can easily observe that the same quantities of interest DFm, ICAm and CAm are obviously higher than in a healthy patient. Use of such a radar chart makes it possible, moreover, to directly compare quantities of interest QI that are intended to be correlated with one another. The production of a multiparameter graphic indicator I in the form of a radar chart makes it possible to visually highlight an increase of the inner surface area of the lumina CLi and more generally of the size of the bronchioli respectively described, with reference to FIG. 7B, by the area ICAm and the Feret diameter DFm.

FIGS. 8 and 9 highlight even further the mean quantities of interest CWWm and CAm substantially identical to the standard quantities of interest, while the mean quantities of interest DFm and ICAm show a marked increase, such mean quantities of interest being characteristic of a patient suffering from chronic SAR.

Moreover, in a variant or in addition, a method 100 for producing a multiparameter graphic indicator I according to the invention can comprise a step 132 for producing the return or graphic output of such a multiparameter graphic indicator I according to the invention by means of any suitable human-machine interface, such as, for example, non-limitatively a computer screen, cooperating with the electronic object implementing said method 100. In a variant or in addition, such return or output can be in writing or acoustic, respectively via an output peripheral of the printer type or also an acoustic output peripheral via a loudspeaker.

In addition to the step 132, a method 100 for producing a multiparameter graphic indicator I according to the invention can comprise one or more steps 131, 133, 134 for producing a graphic rendering respectively of digital representations of the RDI, MRI type and estimated quantities of interest QI in the form of charts. Such charts can be imaged graphically by means of an output peripheral, the same as, or different from, the aforementioned one delivering the multiparameter graphic indicator, quantity of interest by quantity of interest in step 120.

In this way, the user of an electronic object capable of implementing a method according to the invention such as the method 100, has available a plurality of objective, reproducible and instant information aiding the diagnosis of a pathology such as SAR. The set of steps 131 to 134 thus constitute a processing 130, intended to return to the user a multiparameter graphic indicator I, one or more estimated quantities of interest QI, or one or more charts, in this case, one or more digital representations from the aforementioned digital representations RDI, MRI.

Alternatively but non-limitatively, the invention provides for the estimated quantities of interest DFi, CAi, CWWi, ICAi, or more generally a quantity of interest QI, for each type CTi of component Ci, of a digital representation of a histological section of an organ of a patient examined, to be able to be compared not to the corresponding standard quantities of interest in a healthy patient, but to the estimated mean quantities of interest DFm, CAm, CWWm, ICAm in this same patient. According to this variant, the invention provides that one or more steps of graphic rendering 133 or 134 of a method 100 according to the invention can consist moreover of allocating, to the pixels associated with a component of interest Ci, a determined colour and/or value expressing a significant increase or decrease with respect to the mean morphology of the other components.

The invention has been described in particular with reference to analysis of a pulmonary lobe of a mouse. However, it should not be limited to this embodiment example and/or application alone. Other modifications may be envisaged without departing from the scope of the present invention in order to adapt the method, in a variant or in addition, to produce a multiparameter graphic indicator in a human being or another animal, or to an organ having anatomical similarities with the lung.

Furthermore, the invention should not be limited only to graphic representations in the form of the aforementioned bar charts and/or radar charts, wherein the quantities of interest are expressed in particular as a function of standard quantity of interest. In a variant, other representations capable of producing a multiparameter graphic indicator I could have been used.

1. Method for producing a multiparameter graphic indicator relative to a remodelling of the human or animal bronchial epithelium from a digital representation in the form of a matrix of a determined number of pixels, a histological section of a lung, said histological section comprising one or more components having annular shapes each one of which describes a wall encircling a lumen, said method being implemented by a processing unit of a system for histological analysis, said system moreover comprising an output human-machine interface and a data memory, wherein said method comprises:
estimating, from pixels of said digital representation of a histological section of a lung, quantities of interest relative to the respective morphologies of the components identified in said digital representation of the histological section, said quantities of interest belonging to a set of quantities of interest comprising:
i. the Feret diameter of the outer contour of the wall of a component;
ii. the mean distance separating said outer contour from the inner contour (CWICi), revealing a mean thickness of the wall of a component;
iii. the area described by said inner contour of a component, revealing the lumen thereof;
iv. the area described by said outer contour of a component, revealing the total area covered thereby;
v. the area of the wall of a component defined by subtracting the area described by said inner contour from the area described by said outer contour of said component;
producing, per estimated quantity of interest, a graphic representation thereof relative to a standard quantity of interest; and
causing the joint graphic outputting of the graphic representations of said quantities of interest relative to the respective standard quantities of interest produced beforehand, by the output human-machine interface of the system.

2. Method according to claim 1, in which the estimating quantities of interest relative to the respective morphologies of the components identified in the digital representation of the histological section comprises registering in the data memory a data structure associated with each component, said data structure comprising a field for storing the value of each estimated quantity of interest.

3. Method according to claim 2, comprising characterizing a type of component based on the value of one of the estimated quantities of interest, wherein registering in the data memory a data structure associated with each estimated quantity of interest of a component comprises registering in a field of said data structure a value characterizing a determined type of component.

4. Method according to claim 3, in which, when one of the estimated quantities of interest comprises the Feret diameter of the outer contour of the wall of the component, the step for characterizing a type of component comprises an operation of comparison of the value of said estimated Feret diameter to a high-diameter threshold and/or a low-diameter threshold.

5. Method according to claim 3, in which, when one of the estimated quantities of interest comprises the mean thickness of the wall of the component, the step for characterizing a type of component comprises an operation of comparison of the value of said mean thickness to a predetermined high-thickness threshold and/or low-thickness threshold.

6. Method according to claim 3, in which, when one of the estimated quantities of interest comprises the area described by the inner contour of the component, the step for characterizing a type of component comprises an operation of comparison of the value of said area to a predetermined high-area threshold and/or low-area threshold.

7. Method according to claim 3, in which the step for producing, per estimated quantity of interest, a graphic representation thereof relative to a standard quantity of interest and/or the step for producing the joint graphic rendering of the graphic representations of said quantities of interest relative to the standard quantities of interest are only implemented for a determined characterized type of component.

8. Method according to claim 7, for which the type of determined characterized component is a bronchiolus.

9. Method according to claim 1, wherein the step for causing the joint graphic outputting of the graphic representations of said quantities of interest relative to the respective standard quantities of interest produced beforehand by the output human-machine interface of the system, comprises the display by the latter of a radar chart, showing at least three graphic representations of quantities of interest relative to the respective standard quantities of interest on normalized axes.

10. Method according to claim 1, wherein the step for causing the joint graphic outputting of the graphic representations of said quantities of interest relative to the respective standard quantities of interest produced beforehand by the output human-machine interface of the system, comprises the display by the latter of a bar chart, showing the graphic representations of quantities of interest relative to the respective standard quantities of interest by normalized bars.

11. Method according to claim 9, wherein the normalization of an axis or of a bar comprises expressing the value of an estimated quantity of interest as a percentage of the value of the associated standard quantity of interest.

12. Method according to claim 9, wherein the normalization of an axis or of a bar comprises expressing the value of an estimated quantity of interest relative to the value of the associated standard quantity of interest in the form of three predetermined values respectively describing estimated quantity of interest values substantially less than, similar to or greater than, the values of the associated standard quantities of interest.

13. Electronic object of a system for histological analysis, said electronic object comprising a processing unit and cooperating with an output human-machine interface and with a data memory, and wherein said data memory comprises:
a digital representation of a histological section of a human or animal organ; and
instructions that can be executed or interpreted by the processing unit, wherein the interpretation or execution of said instructions by said processing unit causes the implementation of the method according to claim 1.

14. System for histological analysis comprising an electronic object according to claim 13, and an output human-machine interface capable of output to a user a multiparameter graphic indicator according to said method and implemented by said electronic object.

15. Non-transitory computer program product comprising one or more instructions that can be interpreted or executed by a processing unit of an electronic object according to claim 13, wherein the interpretation or execution of said instructions by said processing unit causes the implementation of said method.
